(19) United States
(12) Patent Application Publication
(10) Pub. No.: US 2011/0117152 A1
(43) Pub. Date: May 19, 2011

(54) SILICONE RUBBER EXHIBITING EFFECTIVE ANTIMICROBIAL ACTIVITY

(76) Inventors: Aijun Zhu, Fort Wayne, IN (US); Yi-feng Wang, Clifton park, NY (US)

(21) Appl. No.: 12/919,609

(22) PCT Filed: Mar. 31, 2008
(86) PCT No.: PCT/US2008/004175

§ 371 Date: Nov. 19, 2010

Related U.S. Application Data
(63) Continuation of application No. 12/072,486, filed on Feb. 26, 2008.

(51) Int. Cl. A01N 59/16; A01N 55/02; A01N 25/00; A01N 59/26; A01P 1/00; B82Y 5/00

(52) U.S. Cl. 424/405; 424/618; 514/495; 424/604; 977/810; 977/904

(57) ABSTRACT

A silicone rubber exhibiting antimicrobial efficacy contains: a) at least one silicone rubber derived from a curable silicone rubber-forming composition; b) at least one silver-containing antimicrobial agent incorporated in silicone rubber (a) in X weight percent amount; and, c) at least one carboxylic acid incorporated in silicone rubber (a) in Y weight percent amount, silver-containing antimicrobial agent (b) when incorporated by itself in silicone rubber (a) in an amount of X+Y weight percent therein and carboxylic acid (c) when incorporated by itself in silicone rubber (a) in an amount of X+Y weight percent therein imparting no significant antimicrobial activity to silicone rubber (a) but in the combination therein of up to X weight percent silver-containing antimicrobial agent (b) and up to Y weight percent carboxylic acid (c) imparting significant antimicrobial activity to silicone rubber (a), such activity being exhibited at the interface of an exposed surface of silicone rubber (a) and a microbe-populated aqueous material in contact therewith.

### BACKGROUND OF THE INVENTION

This invention relates to a silicone rubber exhibiting effective antimicrobial activity in a microbe-populated aqueous environment to which a surface of the rubber is exposed.

Silver-containing antimicrobial agents have been incorporated in a variety of synthetic resins in order to provide products which exhibit antimicrobial characteristics. Although silver-based antimicrobial agents may provide suitable antimicrobial properties for some kinds of resins, it has been discovered that silicone rubbers containing silver-type antimicrobial agents often fail to exhibit significant antimicrobial activity. This lack of antimicrobial effectiveness may be due to there being relatively low amounts of silver available at the surface of the rubbers. Whatever the actual cause may be for the observed lack of efficacious antimicrobial activity in silicone rubbers containing known types of silver-based antimicrobial agents, it remains that a need exists for imparting effective levels of antimicrobial activity to silicone rubbers which are intended to utilize silver-containing antimicrobial agents that by themselves are inefficacious.

### SUMMARY OF THE INVENTION

In accordance with the present invention, there is provided a silicone rubber exhibiting antimicrobial efficacy which comprises:
a) at least one silicone rubber derived from a curable silicone rubber-forming composition;
b) at least one silver-containing antimicrobial agent incorporated in silicone rubber (a) in X weight percent amount; and,
c) at least one carboxylic acid incorporated in silicone rubber (a) in Y weight percent amount,
   silver-containing antimicrobial agent (b) when incorporated by itself in silicone rubber (a) in an amount of X+Y weight percent therein and carboxylic acid (c) when incorporated by itself in silicone rubber (a) in an amount of X+Y weight percent therein imparting no significant antimicrobial activity to silicone rubber (a) but in the combination therein of up to X weight percent silver-containing antimicrobial agent (b) and up to Y weight percent carboxylic acid (c) imparting significant antimicrobial activity to silicone rubber (a), such activity being exhibited at the interface of an exposed surface of silicone rubber (a) and a microbe-populated aqueous material in contact therewith.

Although not scientifically demonstrated at this time, it is believed that silver-containing antimicrobial substance (b), which lacks appreciable antimicrobial activity when present by itself in silicone rubber (a), exhibits a significant level of antimicrobial activity when carboxylic acid (c) is additionally present in the rubber due to the ability of the carboxylic acid to increase the availability of antimicrobial silver at the surface of the rubber, perhaps by facilitating or improving the migration of silver from within the rubber to its surface.

Whatever the actual mechanism involved, it remains that silver-containing antimicrobial agents that are known to be effective in a variety of synthetic resins but not in silicone rubber can be rendered efficacious when accompanied by a carboxylic acid.

The expressions “silicone rubber” and “silicone elastomer” are to be regarded herein as synonymous.

The expression “silver-containing antimicrobial agent” shall be understood herein to mean silver in any of its forms that exhibit antimicrobial activity in a microbe-populated aqueous environment and includes metallic silver, e.g., colloidal silver and nanosilver, silver compounds providing silver ions in aqueous media, complexes of silver, and the like.

The expression “carboxylic acid” shall be understood herein to include mono-, di-, tri-, etc., aliphatic, cycloaliphatic and aromatic carboxylic acids and the anhydrides thereof where such exist as well as mixtures of two or more of any of the foregoing.

Other than in the working examples or where otherwise indicated, all numbers expressing amounts of materials, reaction conditions, time durations, quantified properties of materials, and so forth, stated in the specification and claims are to be understood as being modified in all instances by the term “about.”

It will also be understood that any numerical range recited herein is intended to include all sub-ranges within that range and any combination of the various endpoints of such ranges or subranges.

It will be further understood that any compound, material or substance which is expressly or implicitly disclosed in the specification and/or recited in a claim as belonging to a group of structurally, compositionally and/or functionally related compounds, materials or substances includes individual representatives of the group and all combinations thereof.

### DETAILED DESCRIPTION OF THE INVENTION

This invention encompasses a silicone rubber containing at least one silver-based antimicrobial agent that by itself exhibits little if any antimicrobial activity and at least one carboxylic acid in an amount sufficient to potentiate the antimicrobial activity of the silver-based antimicrobial agent.

This invention also encompasses a method of forming a silicone elastomer comprising the steps of providing a curable silicone rubber-forming composition, introducing therein at least one silver-containing antimicrobial agent in an amount of from 0.1 to 10, preferably from 0.2 to 5 and more preferably from 0.5 to 2, percent by weight, and at least one carboxylic acid in an amount of from 0.1 to 5, preferably from 0.2 to 3 and more preferably from 0.5 to 2, percent by weight, and subjecting the silicone rubber-forming composition to curing conditions to provide the silicone rubber.

Silicone rubber (a) can be obtained by curing any one of numerous known curable silicone rubber-forming compositions, e.g., liquid silicone rubber (LSR) compositions, room temperature vulcanizable silicone rubber (RTV) compositions and heat curable silicone elastomer (HCE) compositions.

A typical LSR composition is a multi-component combination of a vinyl-containing polydiorgansiloxane fluid, a hydrogen-containing polydiorganosiloxane fluid, an effective amount of a platinum catalyst and a reinforcing filler such as a fumed silica and one or more additional additives. Typically, two-component LSR mixtures are mixed and charged into a preheated mold where they are rapidly cured to produce a silicone rubber article. A first component, or package, includes a vinyl-containing polydiorgansiloxane fluid, a silica filler and an effective amount of a platinum catalyst and a second component, or package, includes a hydrogen-containing polydiorganosiloxane fluid in combination with other ingredients including a vinyl-containing polyorganosiloxane fluid and a silica filler. Typically, the LSR composition is produced by kneading a polydiorganosiloxane, inorganic filler and additives by means of a kneading machine such as a Banbury mixer, a turbulizer, a change can mixer or a low intensity double arm dough mixer. In this process, polydiorganosiloxane, inorganic filler, treating agents and additives are batch mixed until the desired properties are obtained. The batch mixing process can take 12 to 30 hours per batch. After mixing, the LSR composition is stripped of volatiles and cooled. For additional details regarding LSRs and silicone rubbers obtained therefrom, reference may be made, inter alia, to U.S. Pat. No. 6,444,154, the entire contents of which are incorporated by reference herein.

RTV silicone compositions may be formulated as one-component or two-component systems. A common form of one-component RTV silicone, curable by exposure to moisture, includes an organosiloxane polymer possessing terminal hydroxyl groups, a polyfunctional organosilicone cross-linking agent and a crosslinking catalyst, e.g., as disclosed in U.S. Pat. Nos. 4,100,129, 4,593,085, 5,420,196, 5,932,650 and 6,737,494, the entire contents of which are incorporated by reference herein. Two-component RTV silicone systems typically comprise an “A” package containing a dihydroxy or silanol-terminated polydiorganosiloxane, a semi-reinforcing filler such as calcium carbonate or ground quartz, a reinforcing filler such as fumed silica and water and a “B” package containing a T or Q functional crosslinker and a condensation cure catalyst, e.g., as disclosed in U.S. Pat. No. 4,490,500, the entire of contents of which are incorporated by reference herein.

HCE compositions generally consist of a diorganopolysiloxane gum, fluid, filler and, optionally, a curing agent such as an organic peroxide. For further details regarding HCE silicone elastomers, reference may be made, inter alia, to U.S. Pat. Nos. 6,245,875 and 6,750,279, the entire contents of which are incorporated by reference herein.

The curable silicone elastomer-forming composition can also contain one or more known and conventional additives, e.g., UV stabilizers, antioxidants, colorants, fillers, reinforcing agents, etc., incorporated in the usual amounts.

The silicone rubber of the invention is useful as, or in, many kinds of articles for which efficacious antimicrobial activity is a necessary or desirable property, e.g., medical and diagnostic instruments, catheters, wound care and medical dressing products, scar care management products, drug delivery systems, and the like.

Useful silver-containing antimicrobial agents include silver-containing inorganic compounds such as silver zirconium phosphates available from Milliken & Company under the tradename ALPHASAN™ RC-2000, RC-5000 and RC-7000; silver-containing organic compounds such as silver carboxylates, e.g., silver benzoate, silver citrate and silver tartrate; silver-substituted zeolites available from Shingawa under the tradename ZEOMIC™; silver-containing glasses available from Ishizuka Glass under the tradename IONPURE™; AMP™ T558 and MICROFREE™, both available from DuPont, and JMAC™, available from Johnson Mathey; and, metallic silver materials such as colloidal silver, nanosilver and silver alloys.

Average particle size of the silver-containing antimicrobial agent can vary widely, e.g., from 5 to 30 nm in the case of nanosilver and from 5 microns to 100 microns for all other forms.

Carboxylic acid component (c) as previously defined can be selected from any of numerous known aliphatic, cycloaliphatic and aromatic carboxylic acids possessing one or more carboxylic acid groups, the anhydrides thereof where such exist, and mixtures thereof, provided the presence of a particular carboxylic acid, carboxylic acid anhydride or mixture of particular carboxylic acid(s) and/or carboxylic acid anhydride(s) in a given curable silicone rubber-forming composition does not adversely affect its cure or the properties of the cured rubber. In general, the carbon content of the useful carboxylic acids and carboxylic acid anhydrides will not exceed 14 carbon atoms when the carboxylic acids and anhydrides thereof are of the saturated aliphatic, cycloaliphatic or aromatic variety and in the case of unsaturated aliphatic and cycloaliphatic carboxylic acids and carboxylic acid anhydrides where the presence of one or more olefinic bonds tends to lower the melting point and increase solubility, a carbon content that does not exceed 20 carbon atoms. Some useful carboxylic acids and carboxylic acid anhydrides that can be utilized herein include propanoic acid, 2-methyl propanoic acid, butanoic acid, pentanoic acid (valeric acid), hexanoic acid (caproic acid), 2-ethylhexanoic acid, heptanoic acid (enanthic acid), octanoic acid (caprylic acid), oleic acid, linoleic acid, linolenic acid, cyclohexanecarboxylic acid, cyclohexylacetic acid, cyclohexenecarboxylic acid, benzoic acid, benzeneacetic acid, propanedioic acid (malonic acid), butanedioic acid (succinic acid), hexanedioic acid (adipic acid), 2-butenedioc acid (maleic acid), acetic anhydride, maleic anhydride, and the like.

Comparative Examples 1-16 are illustrative of silicone rubbers containing various silver-type antimicrobial agents by themselves or the aliphatic carboxylic acid oleic acid by itself and as such are outside the scope of the invention. Examples 1-25 are illustrative of the antimicrobially efficacious silicone rubbers of this invention which contains both a silver-type antimicrobial agent and an aliphatic carboxylic acid.

Antimicrobial efficacy was determined by the AATCC 100/Film Contact Method as modified in accordance with the description of this method which follows. The test method provides a quantitative procedure for the evaluation of the degree of antibacterial activity, in this case, test samples of cured silicone rubber samples, or specimens, containing a variety of silver-containing antimicrobial agents, carboxylic acids and mixtures of both.

Silicone rubber film of 2 mm thickness is inoculated with a known concentration of two test organisms, Staphylococcus aureus and Escherichia coli, and thereafter cut into 35 mm×35 mm specimens. A test specimen is placed in a sterile 60 mm petri dish and “floated” in a 100 mm×20 mm petri dish containing sterile water (for humidity purposes). The test specimen is then inoculated with 0.4 mL of the test organism and incubated for 24 hours after which the specimen is neutralized with Letheen broth and serial dilutions are performed. Percentage reduction and/or log reduction is reported along with the zero time count and 24 time count.

### Test Procedures

1. Inoculate SCD broth with the appropriate test organisms (S. aureus and E. coli). Incubate for 18-24 hours.

2. Standardize test organisms with a spectrophotometer to reach a population of 10⁸.

3. Dilute the test organisms with 0.2% Nutrient Broth and sterile saline to obtain an approximate population of 1-5×10⁵.

4. Inoculate the test samples: two 35 mm×35 mm test samples are inoculated with 0.4 mL of each test organism (one for the zero time and one for the 24 hour time).

5. Neutralize the zero time sample with Letheen Broth and perform serial dilutions. Plate with TSA agar and incubate 48 hours at 36-38° C.

6. Incubate the 24 hour “floated” test sample for 24 hours at 36-38° C.

7. After 24 hours, the 24 hour test sample is neutralized with Letheen Broth and serial dilutions are performed. These plates are also incubated 48 hours at 36-38° C.

8. Percent reduction and, optionally, log reduction, are reported.

### Comparative Examples 1-4

These examples show that antimicrobial nanosilver agents, known to be effective by themselves when incorporated into various thermoplastic resins, are essentially inactive when incorporated in a conventional LSR.

Test specimens of a conventional first LSR (Silicone Rubber A) containing nanosilver particles were prepared with the antimicrobial effectiveness of each specimen being evaluated employing the aforedescribed microbiological assay procedure. The results of the assays were as follows:

TABLE 1
Microbiological Test Results for Silicone Rubber A
Containing Nanosilver

| | Anti- | | Initial | 24 Hour | |
|---|---|---|---|---|---|
| | microbial | | Contact | Contact | Percent |
| Comp. | Nanosilver, | | Time (CFU/ | Time (CFU/ | Reduc- |
| Ex. | wt. % | Organism | Sample) | Sample) | tion* |
| 1 | 0.5 | S. aureus | 1.2 × 10⁵ | 1.6 × 10⁵ | no effect |
| 2 | 0.5 | E. coli | 1.2 × 10⁵ | 2.2 × 10⁷ | no effect |
| 3 | 0.5 | S. aureus | 1.4 × 10⁵ | 5.0 × 10⁵ | no effect |
| 4 | 0.5 | E. coli | 1.2 × 10⁵ | 7.5 × 10⁷ | no effect |

*Percent reduction based on initial (0 time) number of bacteria.

### Comparative Examples 5 and 6

These examples demonstrate the ineffectiveness of the antimicrobial silver sodium hydrogen zirconium phosphate RC2000 (Milliken & Company) when incorporated by itself in a conventional second LSR (Silicone Rubber B). Using the aforedescribed microbiological assay procedure, the results of the assays were as follows:

TABLE 2
Microbiological Test Results for Silicone Rubber B Containing
Silver Sodium Hydrogen Zirconium Phosphate RC2000

| | Antimicrobial | | Initial | 24 Hour | |
|---|---|---|---|---|---|
| | Silver | | Contact | Contact | Percent |
| Comp. | RC2000, | | Time (CFU/ | Time (CFU/ | Reduc- |
| Ex. | wt. % | Organism | Sample) | Sample) | tion* |
| 5 | 20 | S. aureus | 1.2 × 10⁵ | 1.3 × 10⁶ | no effect |
| 6 | 20 | E. coli | 1.2 × 10⁵ | 2.2 × 10⁷ | no effect |

*Percent reduction based on initial (0 time) number of bacteria.

### Comparative Examples 7 and 8

These examples illustrate the effects of incorporating a silver ion-containing ceramic matrix carrier (SANITIZED AG, Switzerland) in Silicone Rubber A by itself. As the results in Table 3 show, while 1 wt. % loading of this antimicrobial silver-containing agent was ineffective against S. aureus and E. coli.

TABLE 3
Microbiological Test Results for Silicone Rubber A
Incorporating Silver Ion-containing Ceramic Matrix

| | Antimicrobial | | Initial | 24 Hour | |
|---|---|---|---|---|---|
| | Silver Ion- | | Contact | Contact | |
| | Containing | | Time | Time | |
| Comp. | Ceramic | | (CFU/ | (CFU/ | Percent |
| Ex. | Matrix, wt. % | Organism | Sample) | Sample) | Reduction* |
| 7 | 1 | S. aureus | 3.1 × 10⁵ | 3.3 × 10⁶ | no effect |
| 8 | 1 | E. coil | 1.5 × 10⁵ | 1.4 × 10⁶ | no effect |

*Percent reduction based on initial (0 time) number of bacteria.

### Comparative Examples 9-12

Comparative Examples 7 and 8 were substantially repeated but with Silicone Rubber B and with different loadings of the silver ion-containing ceramic matrix. The test results are set forth in Table 4 as follows:

TABLE 4
Microbiological Test Results for Silicone Rubber B
Incorporating Silver Ion-containing Ceramic Matrix

| | Anti- | | | | |
|---|---|---|---|---|---|
| | microbial | | Initial | 24 Hour | |
| | Silver Ion- | | Contact | Contact | |
| | containing | | Time | Time | |
| Comp. | Ceramic | | (CFU/ | (CFU/ | Percent |
| Ex. | Matrix, wt. % | Organism | Sample) | Sample) | Reduction* |
| 9 | 2 | S. aureus | 1.9 × 10⁵ | 2.5 × 10⁵ | No reduction |
| 10 | 2 | E. coli | 2.0 × 10⁵ | 1.9 × 10⁷ | No reduction |
| 11 | 3 | S. aureus | 1.7 × 10⁵ | 4.6 × 10⁵ | No reduction |
| 12 | 3 | E. coli | 1.5 × 10⁵ | 2.7 × 10⁷ | No reduction |

*Percent reduction based on initial (0 time) number of bacteria.

### Example 1

This example illustrates a silicone rubber, specifically, LSR 2050, exhibiting high surface-available antimicrobial silver in accordance with the invention.

Following the general microbiological assay procedures described above, a specimen of Silicone Rubber A containing nanosilver and oleic acid was evaluated with the following results:

TABLE 5
Microbiological Test Results for Silicone Rubber A
Containing Nanosilver

| | Antimicrobial | | Initial | 24 Hour | |
|---|---|---|---|---|---|
| | Nanosilver + | | Contact | Contact | Percent |
| | Carboxylic | | Time (CFU/ | Time (CFU/ | Reduc- |
| Example | Acid | Organism | Sample) | Sample) | tion* |
| 1 | 0.5 wt. % | S. aureus | 1.5 × 10⁵ | 6.6 × 10³ | 95.60% |
| | nanosilver | | | | |
| | and | | | | |
| | 0.5 wt. % | | | | |
| | oleic acid | | | | |

*Percent reduction based on initial (0 time) number of bacteria.

### Comparative Examples 13 and 14

### Examples 2 and 3

Comparative Examples 13 and 14 show that the aliphatic carboxylic acid oleic acid is not effective as an antimicrobial agent when present by itself in Silicone Rubber A but as shown in Examples 2 and 3, in combination with silver sodium hydrogen zirconium phosphate RC 2000 as the antimicrobial silver-containing agent, provides a high level of antimicrobial effectiveness. Employing the standard microbiological test procedures described above, the tests results were as follows:

TABLE 6
Microbiological Test Results for Silicone Rubber A Containing
Oleic Acid Alone and In Combination With Silver Sodium Hydrogen
Zirconium Phosphate RC 2000

| | Antimicrobial | | | | | | |
|---|---|---|---|---|---|---|---|
| | Silver | Oleic | | Initial Contact | 24 Hour | | |
| | RC2000, | Acid, | | Time | Contact Time | Percent | Log |
| Example | wt. % | wt. % | Organism | (CFU/Sample) | (CFU/Sample) | Reduction * | Reduction ** |
| Comp. | — | 0.8 | S. aureus | 1.5 × 10⁵ | 2.3 × 10⁶ | No effect | No effect |
| Ex. 13 | | | | | | | |
| Comp. | — | 0.8 | E. coli | 1.2 × 10⁵ | 3.6 × 10⁷ | No effect | No effect |
| Ex. 14 | | | | | | | |
| Ex. 2 | 5 | 0.8 | S. aureus | 1.9 × 10⁵ | 1.3 × 10² | 99.91% | 4.2 |
| Ex. 3 | 5 | 0.8 | E. coli | 1.5 × 10⁵ | <1.0 × 10¹ | >99.99% | >6.6 |

* Percent reduction based on initial (0 time) number of bacteria.
** Log reduction is based on the control as the reference.

### Comparative Examples 15 and 16

### Examples 4 and 5

Comparative Examples 13 and 14 and Examples 2 and 3 were substantially repeated but with the amounts of silver ion-containing ceramic matrix indicated in Table 7. As reported in the table, the test results were as follows:

TABLE 7
Microbiological Test Results for Silicone Rubber A Containing
Oleic Acid Alone and In Combination With and Without Silver
Ion-containing Ceramic Matrix

| | Antimicrobial | | | | | | |
|---|---|---|---|---|---|---|---|
| | Silver Ion- | | | | | | |
| | containing | Oleic | | Initial Contact | 24 Hour | | |
| | Ceramic | Acid, | | Time | Contact Time | Percent | Log |
| Example | Matrix, wt. % | wt. % | Organism | (CFU/Sample) | (CFU/Sample) | Reduction * | Reduction ** |
| Comp. | — | 0.8 | S. aureus | 1.5 × 10⁵ | 2.3 × 10⁶ | No effect | No effect |
| Ex. 15 | | | | | | | |
| Comp. | — | 0.8 | E. coli | 1.2 × 10⁵ | 3.6 × 10⁷ | No effect | No effect |
| Ex. 16 | | | | | | | |
| Ex. 4 | 1 | 0.8 | S. aureus | 1.8 × 10⁵ | <1.0 × 10¹ | >99.99% | 5.4 |
| Ex. 5 | 1 | 0.8 | E. coli | 1.8 × 10⁵ | <1.0 × 10¹ | >99.99% | >6.6 |

* Percent reduction based on initial (0 time) number of bacteria.
** Log reduction is based on the control as the reference.

### Examples 6-25

Table 8 sets forth the results of microbiological testing of samples of Silicone Rubber B containing combinations of the silver ion-containing ceramic matrix of the previous examples (“Ag”) with each of several different aliphatic carboxylic acids.

| | | | Initial | 24 Hour | |
|---|---|---|---|---|---|
| | Aliphatic Acid + Ag | | Contact Time | Contact Time | Percent |
| Examples | Combination | Organism | (CFU/Sample) | (CFU/Sample) | Reduction* |
| 6, 7 | 0.41 wt. % | S. aureus | 1.7 × 10⁵ | <1.0 × 10¹ | >99.99% |
| | 2-ethylhexanoic acid, | E. coli | 1.8 × 10⁵ | <1.0 × 10¹ | >99.99% |
| | 1 wt. % Ag additive | | | | |
| 8, 9 | 0.41 wt. % caprylic acid, | S. aureus | 2.1 × 10⁵ | <1.0 × 10¹ | >99.99% |
| | 1 wt. % Ag additive | E. coli | 2.0 × 10⁵ | <1.0 × 10¹ | >99.99% |
| 10, 11 | 0.57 wt. % lauric acid, | S. aureus | 2.1 × 10⁵ | <2.2 × 10⁴ | 89.52% |
| | 1 wt. % Ag additive | E. coli | 1.8 × 10⁵ | <1.0 × 10¹ | >99.99% |
| 12, 13 | 0.80 wt. % oleic acid, | S. aureus | 1.9 × 10⁵ | <1.2 × 10² | 99.94% |
| | 1 wt. % Ag additive | E. coli | 1.6 × 10⁵ | <4.0 × 10¹ | 99.97% |
| 14, 15 | 0.80 wt. % linoleic acid, | S. aureus | 1.9 × 10⁵ | <1.0 × 10¹ | >99.99% |
| | 1 wt. % Ag additive | E. coli | 1.6 × 10⁵ | <1.0 × 10¹ | >99.99% |
| 16, 17 | 0.41 wt. % | S. aureus | 1.9 × 10⁵ | <1.0 × 10¹ | >99.99% |
| | 2-ethylhexanoic acid, | E. coli | 1.5 × 10⁵ | <1.0 × 10¹ | >99.99% |
| | 2 wt. % Ag additive | | | | |
| 18, 19 | 0.41 wt. % caprylic acid, | S. aureus | 1.6 × 10⁵ | <1.0 × 10¹ | >99.99% |
| | 2 wt. % Ag additive | E. coli | 1.2 × 10⁵ | <1.0 × 10¹ | >99.99% |
| 20, 21 | 0.57 wt. %, lauric acid, | S. aureus | 1.7 × 10⁵ | <8.0 × 10¹ | 99.95% |
| | 2 wt. % Ag additive | E. coli | 1.6 × 10⁵ | <2.0 × 10¹ | 99.99% |
| 22, 23 | 0.80 wt. %, oleic acid, | S. aureus | 1.7 × 10⁵ | <1.0 × 10¹ | >99.99% |
| | 2 wt. % Ag additive | E. coli | 1.7 × 10⁵ | <2.5 × 10¹ | 99.99% |
| 24, 25 | 0.80 wt. %, linoleic acid, | S. aureus | 1.8 × 10⁵ | <1.0 × 10¹ | >99.99% |
| | 2 wt. % Ag additive | E. coli | 1.7 × 10⁵ | <1.0 × 10¹ | >99.99% |

As the foregoing experimental results show, the combination of silver-based antimicrobial agents with aliphatic carboxylic acids greatly improves the antimicrobial efficacy of silicone rubbers in which they are incorporated, in some cases from no efficacy to high log reduction. The experimental results also demonstrate that the differences in the composition of the LSR have little if any impact on antimicrobial efficacy.

While the invention has been described with reference to a number of exemplary embodiments, it will be understood by those skilled in the art that various changes can be made and equivalents can be substituted for elements thereof without departing from the scope of the invention. In addition, many modifications can be made to adapt a particular situation or material to the teachings of the invention without departing from essential scope thereof. Therefore, it is intended that the invention not be limited to any particular exemplary embodiment disclosed herein.

1. A silicone rubber exhibiting antimicrobial efficacy which comprises:
a) at least one silicone rubber derived from a curable silicone rubber-forming composition;
b) at least one silver-containing antimicrobial agent incorporated in silicone rubber (a) in X weight percent amount; and,
c) at least one carboxylic acid incorporated in silicone rubber (a) in Y weight percent amount,
silver-containing antimicrobial agent (b) when incorporated by itself in silicone rubber (a) in an amount of X+Y weight percent therein and carboxylic acid (c) when incorporated by itself in silicone rubber (a) in an amount of X+Y weight percent therein imparting no significant antimicrobial activity to silicone rubber (a) but in the combination therein of up to X weight percent silver-containing antimicrobial agent (b) and up to Y weight percent carboxylic acid (c) imparting significant antimicrobial activity to silicone rubber (a), such activity being exhibited at the interface of an exposed surface of silicone rubber (a) and a microbe-populated aqueous material in contact therewith.

2. The silicone rubber of claim 1 wherein X is a weight percent range selected from the group consisting of 0.1 to 10, 0.2 to 5 and 0.5 to 2 and Y is a weight percent range selected from the group consisting of 0.1 to 5, 0.2 to 3 and 0.5 to 2.

3. The silicone rubber of claim 1 wherein the curable silicone rubber-forming composition from which silicone rubber (a) is derived is an LSR, RTV or HCE.

4. The silicone rubber of claim 1 wherein silver-containing antimicrobial agent (b) is at least one member selected from the group consisting of inorganic silver compound, organic silver compound, silver ion-containing glass, silver ion-containing zeolite, silver ion-containing ceramic matrix, colloidal silver, nanosilver and silver alloy.

5. The silicone rubber of claim 1 wherein silver-containing antimicrobial agent (a) is at least one of nanosilver, silver ion-containing ceramic matrix and silver sodium hydrogen zirconium phosphate.

6. The silicone rubber of claim 1 wherein carboxylic acid (c) is at least one member selected from the group consisting of aliphatic carboxylic acid, cycloaliphatic carboxylic acid, aromatic carboxylic acid and anhydrides thereof.

7. The silicone rubber of claim 4 wherein carboxylic acid (c) is at least one member selected from the group consisting of aliphatic carboxylic acid, cycloaliphatic carboxylic acid, aromatic carboxylic acid and anhydrides thereof.

8. The silicone rubber of claim 6 wherein carboxylic acid (c) is at least one aliphatic carboxylic acid selected from the group consisting of propanoic acid, 2-methylpropanoic acid, hexanoic acid, heptanoic acid, octanoic acid, lauric acid, loeic acid, linoleic acid, linolenic acid, cyclohexanecarboxylic acid, cyclohexylacetic acid, cyclohexenecarboxylic acid, benzoic acid, benzeneacetic acid, propanedioic acid, butanedioic acid, hexanedioic acid, 2-butenedioic acid, acetic anhydride and maleic anhydride.

9. The silicone rubber of claim 7 wherein carboxylic acid (c) is at least one aliphatic carboxylic acid selected from the group consisting of propanoic acid, 2-methylpropanoic acid, hexanoic acid, heptanoic acid, octanoic acid, lauric acid, loeic acid, linoleic acid, linolenic acid, cyclohexanecarboxylic acid, cyclohexylacetic acid, cyclohexenecarboxylic acid, benzoic acid, benzeneacetic acid, propanedioic acid, butanedioic acid, hexanedioic acid, 2-butenedioic acid, acetic anhydride and maleic anhydride.

10. The silicone rubber of claim 1 wherein the curable silicone rubber-forming composition from which silicone rubber (a) is derived is an LSR, RTV or HCE, silver-containing antimicrobial agent (b) is at least one member selected from the group consisting of inorganic silver compound, organic silver compound, silver ion-containing glass, silver ion-containing zeolite, silver ion-containing ceramic matrix, colloidal silver, nanosilver and silver alloy, carboxylic acid (c) is at least one member selected from the group consisting of aliphatic carboxylic acid, cycloaliphatic carboxylic acid, aromatic carboxylic acid and anhydrides thereof, X is a weight percent range selected from the group consisting of 0.1 to 10, 0.2 to 5 and 0.5 to 2 and Y is a weight percent range selected from the group consisting of 0.1 to 5, 0.2 to 3 and 0.5 to 2.

11. An article made from, or having as a component thereof, the silicone rubber of claim 1.

12. An article made from, or having as a component thereof, the silicone rubber of claim 2.

13. An article made from, or having as a component thereof, the silicone rubber of claim 3.

14. An article made from, or having as a component thereof, the silicone rubber of claim 4.

15. An article made from, or having as a component thereof, the silicone rubber of claim 6.

16. An article made from, or having as a component thereof, the silicone rubber of claim 10.

17. A process of imparting efficacious antimicrobial activity to a silicone rubber (a) derived from a curable silicone rubber-forming composition which comprises:
a) incorporating in the curable silicone rubber-forming composition X weight percent of at least one silver-containing antimicrobial agent (b) and Y weight percent of at least one carboxylic acid (c); and,
b) curing the curable silicone rubber-forming composition to provide silicone rubber (a),
silver-containing antimicrobial agent (b) when incorporated by itself in the rubber-forming composition in an amount of X+Y weight percent therein and carboxylic acid (c) when incorporated by itself in the rubber-forming composition in an amount of X+Y weight percent therein imparting no significant antimicrobial activity to silicone rubbers (a) obtained from the curing of the rubber-forming compositions but in the combination of up to X weight percent silver-containing antimicrobial agent (b) and up to Y weight percent carboxylic acid in the rubber-forming composition, imparting significant antimicrobial activity to silicone rubber (a) obtained therefrom, such activity being exhibited at the interface of an exposed surface of silicone rubber (a) and a microbe-populated aqueous material in contact therewith.

18. The process of claim 17 wherein X is a weight percent range selected from the group consisting of 0.1 to 10, 0.2 to 5 and 0.5 to 2 and Y is a weight percent range selected from the group consisting of 0.1 to 5, 0.2 to 3 and 0.5 to 2.

19. The process of claim 17 wherein the curable silicone rubber-forming composition from which silicone rubber (a) is derived is an LSR, RTV or HCE.

20. The silicone rubber obtained by the process of claim 17.
